(19) United States
(12) Patent Application Publication
(10) Pub. No.: US 2009/0253215 A1
(43) Pub. Date: Oct. 8, 2009

(54) METHOD FOR CONTROLLING THE FLOW OF LIQUIDS CONTAINING BIOLOGICAL MATERIAL BY INDUCING ELECTRO- OR MAGNETO-RHEOLOGICAL EFFECT

(75) Inventors: Rifat Hikmet, Eindhoven (NL); Reinhold Wimberger-Friedl, Veldhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., Eindhoven (NL)

(21) Appl. No.: 11/721,576

(22) PCT Filed: Dec. 16, 2005
(86) PCT No.: PCT/IB2005/054283

§ 371 Date: Jun. 13, 2007

(30) Foreign Application Priority Data
Dec. 23, 2004 (EP) 04106942.8

(51) Int. Cl. G01N 1/00; G01N 33/00

(52) U.S. Cl. 436/174; 422/61

(57) ABSTRACT

The invention provides a method for controlling or manipulating the flow of a solution or liquid comprising biological material or biomolecules. Therefore, particles are added to the solution or liquid for providing the solution or liquid with rheological properties. The solution or liquid comprising the particles is provided in a microchannel and by applying an electric and/or magnetic field to the microchannel, the flow of the liquid or solution can be controlled.

The present invention relates to a method for controlling or manipulating the flow of a liquid comprising biological material or biomolecules, such as e.g. a bodily fluid, in a microchannel of a microfluidic device, such as e.g. a lab-on-chip device or a biosensor.

A ‘Lab-on-chip device’ is an integrated microfluidic system on a microscale chip, also called microchip. A useful reference for such devices is “Microsystem Engineering of Lab-on-a-Chip Devices, Geschke et al., Wiley, 2004. The microchips may be made of glass, polymers or silicon and may comprise channels, mixers, reservoirs, diffusion chambers, integrated electrodes, pumps, valves, etc. Lab-on-chip systems integrate numerous fluidic operations, such as for example mixing and/or separation, with various read-out modalities for achieving rapid analysis of biochemical or biological reactions in very small, e.g. nanoliter, volumes. In lab-on-chip technology chemical synthesis and/or analysis is carried out in microscopic channels integrated in a cartridge. The main advantages of such systems are the drastically reduced amount of reagent volumes, the high reaction rate due to the short diffusion lengths in the microchannels, the integration of electronic and/or optical sensors and the possibility to make very low-cost disposable devices, which is particularly interesting for biological systems.

Carrying out reactions requires manipulating liquid flows for mixing, filtering, extraction, incubation and/or sensing purposes. In general, the liquid routing is done by a combination of channels, pumps and valves. For a low-cost disposable device it is, however, important to have implementations of the basic functions in low cost technology. Pumping can be done by external forces like pressure, by capillary force, or by force fields like electrical force, gravitational force, etc. For valves, active or passive valves can be used. Active valves comprising moving parts (MEMS) are difficult to make and lack reliability. Passive valves based on interfacial tension are very attractive but only function at the flow front and not anymore in a continuous flow. There is a lack of simple active valves which can be integrated in a disposable cartridge technology.

Electro- or magneto-rheological (ER or MR) fluids are smart materials whose rheological properties (viscosity, yield stress, shear modulus, etc.) can be readily controlled using an external electric or magnetic field. ER or MR fluids can switch from a liquid-like material to a solid-like material within a millisecond with the aid of an electric or magnetic field. This phenomenon is called the ER or MR effect. The unique feature of the ER or MR effect is that ER or MR fluids can reversibly and continuously change from a liquid state to a solid state.

Properties of electro-rheological fluids (ERFs) can be electrically controlled. These electrically controlled rheological properties of ERFs can be used in a wide range of technologies requiring damping or resistive force generation. Examples of such applications may be active vibration suppression and motion control. Several commercial applications have been explored, mostly in the automotive industry, such as, for example, ERF-based engine mounts, shock absorbers, clutches and seat dampers. Other applications include variable-resistance exercise equipment, earthquake-resistant tall structures, and positioning.

In almost all applications of such magneto-rheological (MR) and electro-rheological (ER) liquids, application of a magnetic respectively electric field is used in order to cause an increase in the viscosity of the ER or MR liquid. This effect may be used in pumps and actuators, and in breaks.

The mechanism of the ER or MR effect is intensively discussed in various review articles. A generally accepted concept for the positive ER or MR effect is that the particles form fibrillated chains, which contribute to abrupt increases of the rheological parameters.

Most ER or MR fluids are made of particles dispersed in liquids. FIG. 2 illustrates a channel 8 comprising an ER or MR fluid. Reference numeral 9 illustrates particles present in the ER or MR fluid. FIG. 3 illustrates how, in the presence of an electric or magnetic field, for example an electric field applied to the ER or MR fluid by means of electrodes 10, the particles 9 of the ER or MR fluid form chains along the field lines of the applied field, due to an induced dipole moment. The structure induces changes to the viscosity, yield stress and other properties of the ER or MR fluid, allowing the ER or MR fluid to change consistency from that of a liquid to something that is viscoelastic, with response times to changes in electric or magnetic fields on the order of milliseconds.

The dispersed phase of ER or MR fluids can be either solid (forming a suspension) or liquid (forming an emulsion), with particles which may be ceramics, organics or polymers. The particles' size and shape have an impact on the ER or MR effect. The influence of particle size on the ER or MR effect is quite diverse. Particles of size 0.1 μm to 100 μm are commonly used in the preparation of ER or MR fluids. The ER or MR effect is expected to be weak if the particles are too small, as Brownian motion tends to compete with particle fibrillation. Very large particles are also expected to display a weak ER effect, as sedimentation would prevent the particles from forming fibrillation bridges. It is well known that the dielectric properties of a heterogeneous system largely depend on the geometry of the dispersed particles. Since the ER effect is induced by an external electric field, the dielectric properties of a suspension are believed to play a significant role in the ER effect, as does the geometry of the dispersed particles. Ellipsoidal particles are expected to give a stronger ER effect than spherical particles as the ellipsoidal particles strengthen particle chain formation due to a greater electric-field induced moment. Experimental results show that the dynamic modulus increases almost linearly with the particle geometric aspect ratio (length-to-diameter). An ellipsoidal/spherical blend system shows a much stronger ER or MR effect than a one-component system.

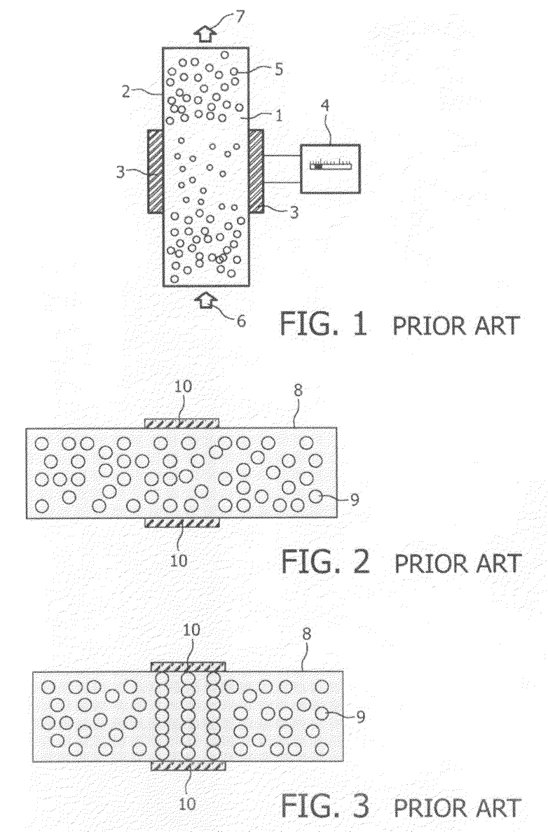

DE 196 13 024 provides a discrete flow controller for controlling the flow of fluids in, for example, food processing. A processing fluid is fed through a vessel containing an added control fluid whose viscosity may be varied by electrical, magnetic or thermal means, as is illustrated in FIG. 1. The vessel 2 contains a control fluid 1 whose viscosity may be influenced by electric or magnetic fields or by temperature changes. The processing fluid in the form of a gas 5 is passed through an inlet and outlet flow (indicated by arrow 6, respectively 7 in FIG. 1) through the vessel 2 at a rate depending on the viscosity of the control fluid 1 which is, for example, varied by means of an electrical or magnetic field applied to electrodes 3 positioned outside (as in FIG. 1) or inside the vessel 2. A control unit 4 adjusts the electrical or magnetic field within the vessel 2 between the electrodes 3.

In DE 196 13 024, the viscosity properties of the control fluid 1, which may be an electro- or magneto-rheological fluid, are used to control the flow-through of the processing fluid 5, and thus the control fluid acts as a discrete valve element. In such valves, the electro- or magneto-rheological fluids are stationary and the flow of processing fluid can be controlled due to the viscosity change.

DE 196 13 024 furthermore provides a method where ER or MR liquid is placed in a device to act as a valve and control the flow of a gas. However the application and integration of such a device in a microfluidic cartridge is rather difficult. One of the problems to overcome is the positioning of one or more of such discrete elements in a microfluidic channel. This is difficult and costly. It has the same difficulty as including any other valve such as piezoelectric, electro magnetic valves. Another problem is associated with the applicability of such valves for controlling the flow of fluids other than gases. The fluid which needs to be transported needs to be dispersed within the electro-rheological fluid. In the case of gases this is not difficult whereas dispersing a liquid, such as a bodily fluid, in an electro-rheological fluid is not easy.

The valve described in DE 196 13 024 can thus not be used as such for controlling the flow-through of liquid such as bodily fluids in microchannels of microfluidic devices, as bodily fluids as dispersing bodily fluids in a ER or MR liquid on microscopic scale while keeping it stable is very difficult.

It is an object of the present invention to provide a method for controlling the flow of solutions or liquids which comprise biological material, such as for example bodily fluids, through microchannels.

The present invention provides a method for controlling or manipulating the flow of a liquid, e.g. a solution, especially an aqueous solution, comprising biological material or biomolecules in a microchannel of a microfluidic device comprising at least one microchannel. The method comprises:
adding particles to the liquid for providing said liquid with rheological properties,
providing the liquid in the microchannel, e.g. by introducing the liquid therein, and
applying an electric and/or a magnetic field to the liquid with the particles.

In a preferred embodiment of the invention, the liquid comprising biological material or biomolecules may be a bodily fluid, such as e.g. blood, blood plasma, urine, interstitial fluid or others.

According to the invention, the particles that are added to the liquid may be compatible with the biological material or biomolecules. The particles may show high resistance toward the biological material or biomolecules, while inducing an electro- or magneto-rheological effect in the liquid.

In an embodiment according to the invention, the microfluidic device may comprise at least two microchannels. The electric or magnetic field may be applied to the liquid at a position in the neighbourhood of a junction between the at least two microchannels.

In embodiments according to the invention, the particles may have a surface and the method may furthermore comprise, before adding the particles to the liquid, modifying the surface of the particles. In some embodiments, modifying the surface of the particles may be performed by providing the particles with a coating which is resistant to the biological material or biomolecules. In other embodiments, modifying the surface may be performed by binding molecules to the surface. The binding of molecules may be performed chemically using, for example, any suitable binding method such as covalent bonding, hydrogen bonding, etc. The binding may be provided, for example by thiol, silane carboxyl groups. The binding may be performed physically using, for example, block copolymers.

In further embodiments of the present invention, the particles which are added to the liquid may induce an electro-rheological effect. In other embodiments, the particles which are added to the liquid may induce a magneto-rheological effect. Examples of the latter may be particles which comprise ferromagnetic materials such as magnetite, manganese ferrite, barium ferrite, iron, cobalt, nickel or iron nitride.

In a specific embodiment of the invention, the particles may be anisometric particles.

The method according to the present invention may for example be used in lab-on-chip devices or in biosensor devices.

The method according to the present invention may e.g. be used in molecular diagnostics, biological sample analysis or chemical sample analysis.

The present invention also provides a kit for carrying out test on a liquid comprising biological material or biomolecules, the kit comprising:
a first receptacle suitable for being filled with particles for providing rheological properties to a liquid,
an inlet for a liquid comprising biological material or biomolecules,
means for allowing said liquid comprising biological material or biomolecules and said particles to be mixed, and
a microfluidic device, the microfluidic device comprising at least one microchannel and a means for applying a magnetic and/or electric field to the at least one microchannel, and
a hygienic seal for preventing contamination of said kit before use.

The kit may furthermore comprise a power source for driving said means for applying a magnetic and/or electric field to the at least one microchannel of the microfluidic device. A second receptacle for being filled with a washing solution may be provided. A means for reading out diagnostic results may also be provided, e.g. directly by means of a microscope or indirectly, e.g. electronic read out.

It is an advantage of the present invention that it enables biological material or biomolecules in liquids to be detected without hindrance by the added particles.

These and other characteristics, features and advantages of the present invention will become apparent from the following detailed description, taken in conjunction with the accompanying drawings, which illustrate, by way of example, the principles of the invention. This description is given for the sake of example only, without limiting the scope of the invention. The reference figures quoted below refer to the attached drawings.

FIG. 1 is a schematic illustration of a device using the rheological effect for controlling the flow-through of a fluid, according to the prior art.

FIG. 2 and FIG. 3 illustrate the orientation of particles in a rheological fluid under the influence of an electric or magnetic field, according to the prior art.

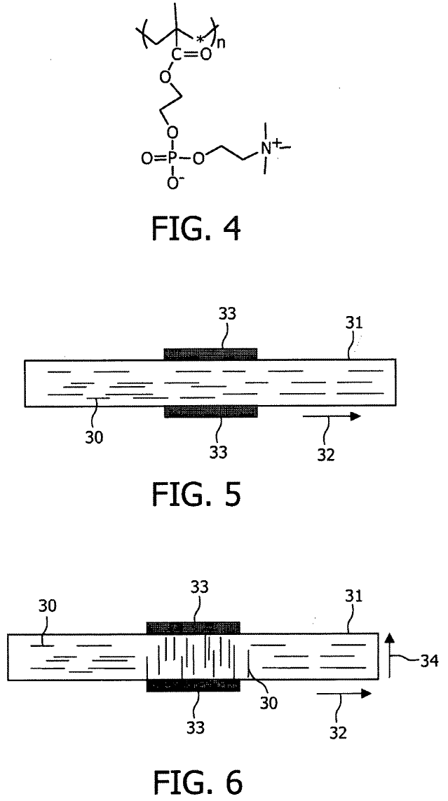

FIG. 4 illustrates an example of a charge neutral salt which may be used in an embodiment of the invention.

FIG. 5 and FIG. 6 illustrate the influence of an electric or magnetic field to the orientation of anisometric particles in a fluid, according to an embodiment of the present invention.

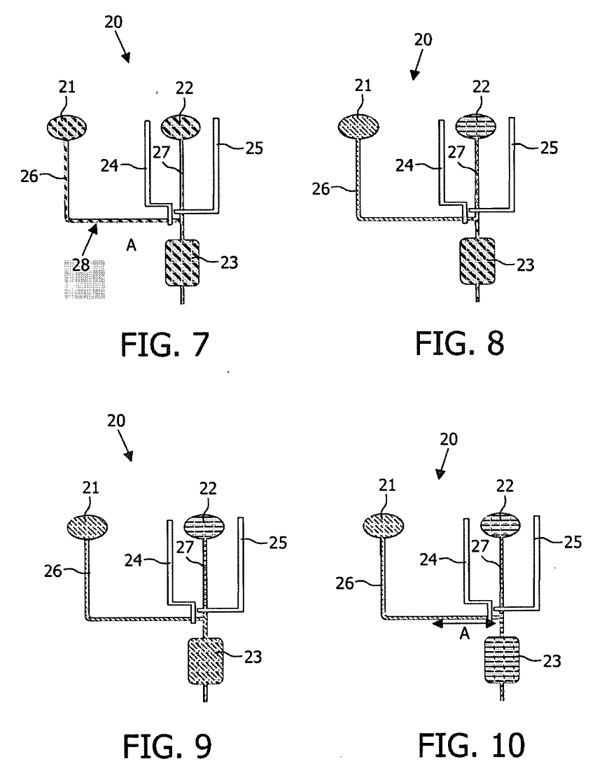

FIG. 7 to FIG. 10 illustrate the working of a microfluidic device comprising a bodily fluid with electro- or magneto-rheological particles, according to embodiments of the present invention.

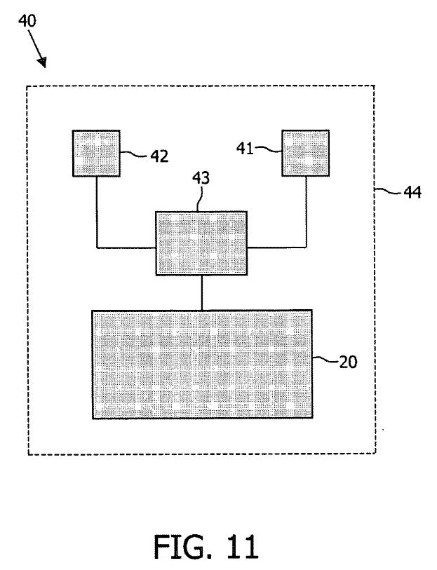

FIG. 11 shows a fluidic device in accordance with a further embodiment of the present invention.

In the different figures, the same reference signs refer to the same or analogous elements.

The present invention will be described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

The present invention provides a method for enhancing rheological properties of bodily fluids, such as blood, blood plasma, urine, interstitial fluid or others, to be analysed in microscopic channels or microchannels in microfluidic devices such as, for example, lab-on-chip devices or biosensors, for the detection of target molecules, such as, for example, antibodies, nucleic acids (e.g. DNR, RNA), toxins, proteins, peptides, oligo- or polysaccharides or sugars, in these bodily fluids.

For example the flow rate of bodily fluids to be analysed in a microchannel of a microfluidic device may be controlled by means of the rheological effect. This rheological effect in bodily fluids may be achieved according to the present invention by adding electro-rheological (ER) or magneto-rheological (MR) particles to the bodily fluids themselves, rather than to a control fluid.

Thus, according to the present invention, a fluid comprising a biological component to be analysed, for example a bodily fluid, such as e.g. blood, blood plasma, urine, interstitial fluid, or other, is provided. With analysed is meant the detection of at least one target molecule or target composition such as, for example, antibodies, toxins, proteins, peptides, lipids, membranes, glycoproteins, cells such as pathogens, lymphatic cells, blood cells. In the further description, the fluid comprising a biological component will be referred to as bodily fluid, especially an animal or human bodily fluid. It has to be understood that the invention is not limited hereto, but that the invention may also be applied to other fluids comprising a biological component. Hereto, a bodily fluid sample, for example a blood sample, is taken from e.g. a human being or an animal. Then, in a first step, ER or MR particles are added to and mixed with the bodily fluid sample. A right choice for ER or MR particles needs to be made before adding them to the bodily fluid sample. Therefore, a number of conditions are preferably taken into account and preferably have to be fulfilled. The conditions, the ER or MR particles preferably have to comply with, will be discussed hereinafter.

The present invention thus makes use of additives, i.e. ER and/or MR particles or EMR particles, which can change the property of a liquid that needs to be analysed so that its flow properties can be controlled or can be controlled better. According to the present invention, the addition of these extra components, i.e. electro- and/or magneto-rheological particles, serves for controlling the flow properties of the fluids that comprise a biological component, for example, bodily fluids, and which need to be analysed.

One factor to take into account is the surface nature of the ER or MR particles that are added to the bodily fluid. When biological molecules come in contact with such ER or MR particles, they might, for example, get adsorbed on the surfaces of the rheological particles. This usually leads to a decreased mobility of the biological molecules or biomolecules to be analysed in the fluid, which may hinder their correct detection.

Hence, additives used to achieve an ER or MR effect in a bodily fluid preferably do not show a high interaction with the biomolecules present in the fluid. Preferably, the additives are bioorthogonal with respect to at least one biomolecule in the bodily fluid. This bioorthogonality implies that the surface of the ER or MR particles should have a high resistance to biomolecules such that the biomolecules do not adsorb to the surface of the ER or MR particles. By applying a suitable surface treatment, adsorption of biomolecules to the surface of the ER or MR particles can be reduced to a large extent.

For example, surfaces of ER or MR particles with one or more of the following structural features show high resistance to biomolecules:
Hydrophilic particles
Particles which are overall electrically neutral
Particles having hydrogen bond acceptors, but no hydrogen bond donors
Molecules comprising one or more groups selected from a hydroxy, alkoxy, amine, alkyl substituted amine, carboxy amine, anhydride urethane and urea are suitable.

The additives may be biocompatible. Biocompatibility refers to the additives not affecting functional domains of the biomolecules which might impede or prevent detection.

Various salts where the anion and cation are bound to the same molecule forming a charge neutral system are also suitable molecules which show good resistance to biomolecules.

A specific example of a charge neutral salt is shown in FIG. 4.

In embodiments of the invention, the surface of the ER or MR particles may be modified in order to prevent adsorption of target molecules to be detected. In one embodiment according to the present invention, this may be done by, for example, providing a coating on the surface of the ER or MR particles. The coating may have one or more of the following structural features to show high resistance to biomolecules as recited above.

A specific example of a molecule which may be used for the modification of the surface of the ER or MR particles may be ethylene oxide poly/oligomers, which work effectively. Other classes of molecules which may be used according to the invention may be molecules with hydroxy groups such as alcohols and sugars, which also appear to be very suitable.

A specific example may, for example, be thiol of ethylene glycol SH—(CH2CH2O)₆—CH3 which effectively works as a bio-resistant molecule, which means that no biomolecules adsorb to a surface modified with this molecule. In the same way a block copolymer comprising polystyrene (e.g. mol weight 3000) and polylene oxide (e.g. mol weight 1000) blocks the adsorption of biomolecules to the surface of ER or MR particles when the surface of these particles is modified with the mentioned block copolymer.

Furthermore, molecules with thiol silane end groups may also be used to modify the surface of ER and MR particles according to the invention. Silane can react with the OH groups on the surface of the MR or ER particles, while thiol groups are known to react with various metal surfaces such as gold and silver. Block copolymers with polar and apolar parts may also be used. The ER or MR particles may be coated with such copolymers through which the apolar part of the polymer gets attached to the ER or MR particle surface and the polar part may form the outer surface of the coated ER or MR particle showing high resistance to biomolecules.

Microscopic channels or microchannels in microfluidic devices such as, for example, lab-on-chip devices or biosensors, may typically have widths and heights in the order of between some tens of micrometer and one millimeter and may have a length of several millimeters. Fluids flow through microchannels at rates in the order of 0.1 to 1 mm/s. Depending on the size of the microchannels, the ER or MR particle size may be selected to be in the range of between 0.1 and 100 μm. When inorganic particles are used with high density, the size of the particles may most preferably be between 0.1 and 1 μm in order to avoid sedimentation problems. When organic particles and composites are used, density differences may be much smaller so that larger particles may also be used. In that case the preferred particle size may be between 1 and 10 μm.

Furthermore, the ER or MR particles used can preferably be such that they do not alter characteristics, such as mobility, of the biological molecules present in the fluid to be analysed, such as e.g. a bodily fluid. As the concentration of the biomaterials to be detected in the bodily fluid might be very small and as only a small amount, typically in the range between 1 μl and 1 ml, of bodily fluid might be available, the internal mobility of the biomolecules which are to be detected is not to be reduced to a large extent by adding the ER or MR particles. If the ER or MR particles added would decrease the mobility of the biomolecules to be detected or immobilise the biomolecules completely by, for example adsorption, then the biomolecules present in the fluid can not be detected correctly. Therefore, the ER or MR particles are preferably such that target molecules to be detected do not adsorb on the surface of the ER or MR particles, as this would give rise to a decreased amount of target molecules detected and hence to an incorrect result of the test that has been carried out. Adsorption can also lead to agglomeration, and particles and molecules settling out of the solution or flocculating. This may cause problems for the bodily fluid to flow through the microchannels and in the worst case, this may cause a blockage inside the microchannels.

Another problem that can arise is sedimentation of the ER or MR particles. The ER or MR particles are preferably chosen such as to prevent their sedimentation. If the ER or MR particles would precipitate inside the microchannels, they could interfere with the target molecules to be detected at positions of e.g. sensors present on the microfluidic device, e.g. a lab-on-chip device or a biosensor. Again, the surface of the added particles should be biocompatible and/or bioorthogonal with the biomolecules. This biocompatibilisation or biorthogonalisation of the surface of the particles may have the positive influence of decreasing the sedimentation problems of the particles, hence keeping them mobile in the liquid.

Furthermore, also the concentration of ER or MR particles that are added to the bodily fluid sample can be of importance. The concentration or density of ER or MR particles should not be too high, because otherwise the too high pressures may arise in the microchannels. This is most critical in systems where the flow of a fluid is driven by capillary forces. For example, capillary forces of a water-based solution may give a pressure drop in the order of e.g. 50 mbar. This may be sufficient to transport water with a velocity of 1 mm/s through a channel with a height of 50 μm and a length of 1 m. By changing the viscosity of the water-based solution, the velocity will change inversely. According to the invention, the viscosity changes may be less than a factor 100 and may preferably be less than a factor 10 with respect to the as received biological fluid.

In the literature, various ER or MR particles are described. In the method according to the present invention, suitable ER particles are selected which are applicable in systems with low ionic conductivity. In the case of MR, the presence of conductive species, e.g. water is not a problem. In accordance with an aspect of the present invention care is taken in order to make the surfaces of the materials resistant to biomolecules.

Hereinafter, two classes of materials are described by way of example which may be used according to the present invention for inducing ER and MR effects in fluids comprising biological material although the present invention is not limited to these two.

A first class of materials can be used as well for introducing the ER as for introducing the EM effect. This class of materials is called anisometric particles. Anisometric particles with a high aspect ratio show electro-rheological effect. They may be rod-like, disc-like or have any other suitable, possibly random, shape. However they preferably have a aspect ratio in the range 5-100. In the absence of an electric field (see FIG. 5) the long axis of the particles, indicated by horizontal stripes 30 is aligned in the direction of the bodily fluid flow in a microchannel 31, which is indicated by arrow 32 in FIG. 5. Upon application of an electric field between two electrodes 33, the electric field having a direction indicated by arrow 34 in FIG. 6, and in the example illustrated being substantially perpendicular to the flow direction of the bodily fluid, the flow in the direction of arrow 32 is stopped as the anisometric particles align their long axis, which are indicated by vertical stripes 30 in FIG. 6, in the direction of the electrical field lines, i.e. in the example illustrated perpendicular to the direction of the flow.

Anisometric particles can be produced according to various methods without good shape control and with a large distribution of sizes. One method is based on the evaporation of a thin layer of the material the particles should be formed of on top of a substrate covered with a release coating, followed by the release of that thin layer and “milling” the thin layer to small particle sizes. Other methods include the use of naturally occurring minerals such as, for example, mica, which can also be milled. Silicon and aluminium particles may be produced in liquids. The above materials have random shapes and dimensions. Particles having a specific size and shape may also be produced using lithographic methods such as offset printing, micro contact printing and inkjet printing. In all of these techniques, except for inkjet printing, a patterned surface or a surface to which ink has been transferred in a patterned way (for example, by use of a stamp) is used to transfer ink to another surface comprising a layer to be patterned. The ink may be used as a positive or negative etch resist, depending on the type of ink. If it is used as a negative etch resist, material of the layer to be patterned can be removed selectively by etching from those areas that are not covered or modified by the ink. If the ink is used as a positive etch resist, a second layer of ink providing a higher etch resistance is applied only to the so far unmodified areas of the surface (e.g. by deposition via self-assembly from solution). In this case, in the subsequent etching step, material is removed from those areas that had been modified with the first ink (the one with the lower etch resistance). Other inking-etching schemes are also possible, including the local (patterned) chemical modification of the ink already deposited on the surface.

For introducing the ER effect, the anisometric particles may be formed of, for example, a dielectric material such as a polymer, a ceramic; a metallic material or a semiconductor. It may also be a composite material or it may be a layered system. In order to obtain the MR effect using anisometric particles, the particles preferably comprise a magnetic layer or magnetic particles such as, for example, magnetite, manganese ferrite, barium ferrite, iron, cobalt, nickel, permalloy, iron nitride.

A second class of materials that may be used according to the invention are MR materials which do not need to be anisometric. Their size may be in the range between 0.1 and 10 μm. These materials react to an applied magnetic field. Ferromagnetic particles, such as magnetite, manganese ferrite, barium ferrite, iron, cobalt, nickel, permalloy, iron nitride, or particles coated with such ferromagnetic materials or forming a composite material with them can induce MR effect without the particles needing to be anisometric in shape. Small particles comprising ferromagnetic materials or coated with ferromagnetic materials are readily available or they can be prepared with ease.

For analysing the bodily fluid by means of a microfluidic device, such as e.g. a lab-on-chip device or a biosensor, the bodily fluid with added ER or MR particles is applied to a microfluidic device such as, for example, a lab-on-chip device or a biosensor, for analysing the bodily fluid. The functioning of a microfluidic device according to an embodiment of the invention will hereinafter be explained by means of an example. However, this example is only for the ease of explanation and is not limiting to the invention.

FIG. 7 to 10 illustrate the functioning of a microfluidic device 20 according to a specific example, the microfluidic device 20 comprising a bodily fluid sample with ER or MR particles added to it according to the invention. The microfluidic device 20 may comprise a first and second fluid compartment or reservoir 21 respectively 22, a measurement compartment 23 and a first and second electrode set 24 respectively 25. At first, as illustrated in FIG. 4, the first and second fluid compartments or reservoirs 21, 22 may be filled with air and may comprise the ER or MR particles in a dry state, i.e. not in solution. Then, the first fluid compartment 21 may be filled with the bodily fluid. If ER or MR particles are present in the fluid compartment 21, then bodily fluid may be added and mixed with the ER or MR particles. If not, ER or MR particles are added to the bodily fluid before introducing it into the first fluid compartment 21. The second fluid compartment 22 may be filled with a washing solution, for example with a phosphate buffer solution which may typically be used in biological assays. The fluids, i.e. the bodily fluid with ER or MR particles and the washing solution, are sucked into microchannels 26 respectively 27 directly upon application by means of capillary suction.

By applying, by means of electrode sets 24, 25 an electric field or a magnetic field over the microchannels 26, 27, depending on whether ER respectively MR particles have been added to the bodily fluid sample, the fluid flow stops at the position of the first and second electrode sets 24, 25. The electric or magnetic field may be applied by means of an on-chip or off-chip electric or magnetic field generating means. One condition to be fulfilled is that the electric or magnetic field generating means should be positioned close to the microchannels 26, 27, such that at least one part of the generated electric or magnetic field is positioned across the microchannel 26, 27. Preferably, the electrodes are not in direct contact with the liquid in order to avoid electrolysis. The electrode sets are preferably placed at a position across the channel in a direction representing the smallest distance between two channel walls location at opposite sides of the channel. For manufacturing reasons, preferably, the electrode sets 24, 25 may be integrated in the substrate and/or cover plate, e.g. on the backside. Preferably, the electric or magnetic field is applied to the liquid in the microchannel 26, 27 at the position of a junction between two microchannels 26, 27. In that way, at the moment the electric or magnetic field is applied to the microchannel 26, 27, the flow-through of the fluid is stopped immediately. Fluid routing in a microfluidic device may be performed by applying electric or magnetic field generating means at (inside or outside the microchannel) or near the walls of the microchannels of the microfluidic device. In the example given, illustrated in the drawings, ER particles are added to the bodily fluid and an electric field may be applied by means of an on-chip electric field generating means, i.e. by means of electrode sets 24, 25. For example, when a voltage is put over the first electrode set 24 and over the second electrode set 25, as is illustrated in FIG. 8, an electric field is applied over respectively the first microchannel 26 and the second microchannel 27. Through this, both the flow of the bodily fluid sample with, in the example given, ER particles and the flow of the washing solution are stopped in microchannels 26, 27 at the position of electrode set 24 respectively electrode set 25.

When, in a next step which is illustrated in FIG. 9, the voltage over the first electrode set 24 is removed while the voltage over the second electrode set 25 is kept, the measurement compartment 23 can be filled with the bodily fluid sample while the flow of the washing solution is still being stopped at the position of the second electrode 25. As long as the voltage over the first electrode set 24 stays off, the bodily fluid sample flows through the measurement compartment 23 and tests, such as determination of particular target molecules (e.g. antibodies, peptides, lipids, . . . ) can be performed onto the bodily fluid sample.

When a test is done and no more bodily fluid is required in the measurement compartment 23, the voltage over the first electrode set 24 is switched on again and the flow of the bodily fluid sample is stopped at the position of electrode set 24. Subsequently, the voltage over the second electrode set 25 is turned off. The washing solution now flows through the measurement compartment 23 wherein it may have a cleaning function. This is illustrated in FIG. 10. After that, another test or measurement step can be performed on a further portion of the available bodily fluid sample.

The above-described method allows a convenient application of fluids without immediate uncontrolled start of the sequence. By switching off the voltage over the first electrode set 24 and over the second electrode set 25, the bodily fluid sample respectively the washing solution may be introduced in the measurement compartment 23 at the desired sequence, for example, as described in the above example, alternately. The time of flow-through of a fluid may be determined by the time a voltage is put over the electrode set 24 and/or 25. A control unit for controlling this time of flow-through may be provided.

It is considered that there is no limitation to the complexity of the circuit that can be built in this way. The driving force for the fluid can be capillary suction as long as there is still empty volume or it can be a pressure applied to the fluid at the entrance. For the flow sequence the pressure does not need to be controlled precisely, which largely simplifies the interface between the cartridge in which the microchannels 26, 27 are positioned and the instrument which controls and performs the measurement. It has to be noticed that the above measuring method is only meant as example and is not limiting for the invention. Instead of adding ER particles and applying an electrical field, the through-flow of the bodily fluid may be controlled by adding MR particles to it and then applying a magnetic field to the solution or fluid in microchannels 26, 27.

Furthermore, a microfluidic device may comprise more than two microchannels 26, 27 each having a fluid compartment or reservoir 21, 22 at their entrance, wherein, for example, one of these fluid compartments or reservoirs 21, 22 may comprise a washing solution and the other fluid compartments or reservoirs 21, 22 may comprise the same or different bodily fluid samples each comprising ER and/or MR particles. Either the bodily fluid samples in the different fluid compartments or reservoirs 21, 22 may comprise the same or, preferably, may comprise different ER or MR particles. Through this, it is possible to test different bodily fluid samples at the same time, by which the measurement compartment 23 may be cleaned in between different tests by means of the washing solution.

In other embodiments according to the invention, a magnetic field may be applied as well an electric, either separately or simultaneously to the solution or fluid in microchannels 26, 27. Synergetic effects are usually involved, and the observed electro-magneto-rheological (EMR) effect is stronger than the electro-rheological (ER) effect if only an electric field is applied or the magneto-rheological (MR) effect if only a magnetic field is applied. Herefore, electro-magneto-rheological (EMR) materials are to be added to the bodily fluid sample to be analysed.

In a specific example of such EMR materials, a thin film is prepared by successive evaporation of gold, iron and gold on top of a polymeric layer. The total thickness of the gold/iron/gold layer may be 100 nm. According to this specific example, the gold/iron/gold layer is then stripped of from the polymeric layer by dissolving this polymeric layer and subsequently cut the gold/iron/gold layer into small pieces, for example 1 to 10 μm pieces, e.g. 5 μm pieces. The outer gold surface is reactive and it is subsequently modified, e.g. by, in the specific example given, using polyethylene glycol thiol. This makes the surface of the particles extremely resistant to biomolecules. Subsequently, the particles are mixed into 1 mg/mL of liquid a labelled protein which may, in the example given, be BSA-FITC (albumin, fluorescein isothiocyanate conjugate bovine), obtainable from Sigma, in 0.01M PBS buffer (pH ≈7.3) as a model protein. The concentration of the particles may be 1%. MR effect was obtained by applying and removing a magnetic field, the alignment of the particles could thus be altered, thus changing the flow properties of the liquid the particles are added to. In the same way electric field can be applied across the sample in order to obtain ER effect.

In other embodiments according to the invention, the measurement compartment 23 may be used for mixing fluids. For example, a bodily fluid may be required to be mixed with a particular testing liquid, for example, an analysis buffer for the extraction of DNA or a liquid comprising labels which attach to the biomolecules to be detected in the liquid, in order to be able to perform a particular test on the bodily fluid. Therefore, using a method according to the invention, first an electric or magnetic field may be applied to the second microchannel 27 comprising the particular liquid while no electric nor magnetic field is applied to the microchannel 26 comprising the bodily fluid. In that way the bodily fluid may flow into the measurement compartment 23. When a desired amount of bodily fluid is in the measurement compartment 23, the electric or magnetic field over the second microchannel 27 is turned off and an electric or magnetic field is applied over the first microchannel 26, that way stopping the flow-through of bodily fluid and starting flow-through of the testing liquid. Then, the electric or magnetic field over the second microchannel 27 may be turned back on while the electric or magnetic field over the first microchannel 26 is kept on. No more fluid is entering the measurement compartment 23, and the bodily fluid can now be mixed with the testing liquid before testing is performed.

The present invention furthermore provides a device such as a kit or cartridge 40 for performing tests on a fluid comprising biological material or biomolecules, the kit 40 comprising a microfluidic device 20, comprising at least one microchannel 26, 27 and a means for applying an electric and/or magnetic field to the microchannels 26, 27. The kit 40 is illustrated in FIG. 11. The kit 40 comprises a receptacle 41, which is suitable to be filled with particles (as specified above), e.g. particles in a suitable fluid, and then may act as a source of particles to be added to the liquid comprising biological material or biomolecules. The kit may be supplied with the particles already in the receptacle 41. The kit 40 furthermore comprises an inlet 42 for liquid comprising biological material or biomolecules, such as, for example, a bodily fluid, e.g. blood. The inlet may be such that the analyte liquid is drawn into the device by capillary action. Furthermore, the kit 40 comprises a means 43 for allowing the particles to be mixed with the liquid comprising biological material or biomolecules and which forms a connection between receptacle 41 and inlet 42. The liquid comprising the particles may then enter into the microfluidic device 20 in which the liquid is analysed. The flow of the liquid comprising the particles and being present in the at least one microchannel 26, 27 of the microfluidic device 20 may be controlled according to the method of this invention.

Receptacle 41, inlet 42, means 43 for allowing the particles to be mixed with the liquid comprising biological material or biomolecules and the microfluidic device 20 are, as illustrated in FIG. 11, sealed with a hygienic seal 44 to prevent contamination of the kit 40 before use.

Before the kit 40 is ready to use, a power source (not shown in FIG. 11) may be connected to or added to the kit 40. The power source may, for example, be connection to a mains supply or a battery and should have enough power to drive the means for applying an electric and/or magnetic field to the microchannels 26, 27 of the microfluidic device 20. The hygienic seal 44 may then be broken and the analyte liquid is applied to the inlet 42. For example, a drop of the liquid which has to be analysed, for example blood, may then be provided at inlet 42 and, for example, a button (not shown in the figure) may be pressed to start the flow of the liquid from the inlet 42 and to start the flow of the particles from the receptacle 41 toward means 43 for mixing the particles with the liquid to be analysed. The liquid-particle mix may then enter the microfluidic device 20 to be analysed. The power source may drive means for applying an electric and/or magnetic field to the microchannels 36, 27 of the microfluidic device 20 in order to control the flow of the liquid through the microchannels 26, 27 and the required analyse may then be carried out.

The kit 40 may furthermore comprise means for reading out the diagnostic results (not shown) of the analyse that has been carried out.

In some embodiments according to the invention, the kit 40 may comprise at least one further receptacle which may comprise a washing solution. By controlling the application of the electric and/or magnetic field to the microchannels 26, 27, the flow of the liquid to be analysed and the flow of the washing solution may alternatively be controlled in order to carry out more than one test at the same liquid that has been introduced to the kit 40.

It is an advantage of the present invention to have a method for controlling the flow-through of bodily fluids in microchannels without the need of valves, which require larger space and more complicated structure of the microfluidic devices that are used for analysing and/or determining target molecules in bodily fluids.

It is to be understood that although preferred embodiments, specific constructions and configurations, as well as materials, have been discussed herein for devices according to the present invention, various changes or modifications in form and detail may be made without departing from the scope and spirit of this invention.

1. A method for controlling or manipulating the flow of a liquid comprising biological material or biomolecules in a microchannel (26, 27) of a microfluidic device (20), the microfluidic device (20) comprising at least one microchannel (26, 27), the method comprising:
adding particles to the liquid for providing said liquid with rheological properties,
introducing said liquid into said microchannel (26, 27), and
applying an electric and/or a magnetic field to said liquid (26, 27).

2. A method according to claim 1, wherein said liquid is a bodily fluid.

3. A method according to claim 2, wherein said bodily fluid is one of blood, blood plasma, urine or interstitial fluid.

4. A method according to claim 1, wherein said particles are biocompatible and/or bioorthogonal with said biological material or biomolecules.

5. A method according to claim 1, the microfluidic device (20) comprising at least two microchannels (26, 27), wherein said electric or magnetic field is applied to the liquid at a position in the neighbourhood of a junction between the at least two microchannels (26, 27).

6. A method according to claim 1, said particles comprising a surface, wherein the method furthermore comprises:
modifying said surface of said particles before adding the particles to the liquid.

7. A method according to claim 6, wherein modifying the surface of said particles is performed by providing said particles with a coating which is resistant to said biological material or biomolecules.

8. A method according to claim 6, wherein modifying the surface of said particles is performed by binding molecules to said surface.

9. A method according to claim 1, wherein the particles induce an electro-rheological effect.

10. A method according to claim 1, wherein the particles induce a magneto-rheological effect.

11. A method according to claim 10, wherein the particles comprise ferromagnetic materials such as magnetite, manganese ferrite, barium ferrite, iron, cobalt, nickel or iron nitride.

12. A method according to claim 1, wherein said particles are anisometric particles.

13. A method according to claim 1, wherein said microfluidic device is a lab-on-chip device or a biosensor device.

14. The use of the method according to claim 1 in molecular diagnostics biological sample analysis, or chemical sample analysis.

15. A kit (40) for carrying out test on a liquid comprising biological material or biomolecules, the kit (40) comprising:
a first receptacle (41) suitable for being filled with particles for providing rheological properties to a liquid,
an inlet (42) for a liquid comprising biological material or biomolecules,
means (43) for allowing said liquid comprising biological material or biomolecules and said particles to be mixed, and
a microfluidic device (20), the microfluidic device (20) comprising at least one microchannel (26, 27) and a means for applying a magnetic and/or electric field to the at least one microchannel (26, 27), and
a hygienic seal (44) for preventing contamination of said kit (40) before use.

16. A kit (40) according to claim 15, furthermore comprising a second receptacle for being filled with a washing solution.

17. A kit (40) according to claim 15, furthermore comprising a means for reading out diagnostic results.
